(19) United States
(12) Patent Application Publication
(10) Pub. No.: US 2026/0021062 A1
(43) Pub. Date: Jan. 22, 2026

(54) ARACHIDONYL ETHANOLAMIDE COMPOSITION

(71) Applicant: NANJING NUTRABUILDING BIO-TECH CO., LTD., Nanjing (CN)

(72) Inventors: Ronghua YI, Nanjing (CN); Yongxiao ZHU, Nanjing (CN); Xi ZHU, Nanjing (CN); Qilin LIAO, Nanjing (CN)

(21) Appl. No.: 19/341,866

(22) Filed: Sep. 26, 2025

Related U.S. Application Data
(63) Continuation of application No. PCT/CN2024/082430, filed on Mar. 19, 2024.

(30) Foreign Application Priority Data
Mar. 28, 2023 (WO) PCT/CN2023/084303

(51) Int. Cl. A61K 31/164; A61P 25/24

(52) U.S. Cl. CPC A61K 31/164; A61P 25/24

(57) ABSTRACT

The invention discloses an Anandamide composition, which comprises, by weight, 30-75 parts of Anandamide, 5-15 parts of Linolcoylethanolamide, 2-18 parts of Oleoylethanolamide. The Anandamide composition of the invention can achieve a good antidepressant effect.

### CROSS REFERENCE TO RELATED APPLICATION

This application is a continuation application of International Patent Application No. PCT/CN2024/082430, filed on Mar. 19, 2024, which claims the priority of PCT Application No. PCT/CN2023/084303, filed on Mar. 28, 2023, the contents of all of which are incorporated herein by reference in their entirety.

### FIELD OF THE INVENTION

The invention relates to the technical field of health food and dietary supplements, and particularly relates to an Anandamide composition comprising, by weight, 30-75 parts of Anandamide, 5-15 parts of Linoleoylethanolamide, 2-18 parts of Oleoylethanolamide.

### BACKGROUND OF THE INVENTION

Anandamide, also known as Arachidonyl ethanolamide or Arachidonylethanolamide (AEA), is an endogenous cannabinoid neurotransmitter. The endocannabinoid system is an important lipid signaling system that emerged before the evolution of vertebrates and it is well preserved across species. In mammals, it has been recently recognized as a modulator of a large variety of physiological processes, including inflammation and pain, appetite, mood, and pre-and postnatal development. Furthermore, the endocannabinoid system is an important constituent of the neuronal substrates involved in the reinforcement and reward processes of the brain. There are few studies to research AEA by oral administration can improve mood, relieve stress and make people happy.

Young people nowadays are stressed and often feel anxious, the AEA composition may be a potential substance that can relieve anxiety and improve mood, and there will be a broad market demand.

### SUMMARY OF THE INVENTION

In one aspect, the invention provides an Anandamide composition comprising, by weight, 30-75 parts of Anandamide (AEA), 5-15 parts of Linoleoylethanolamide (LEA), 2-18 parts of Oleoylethanolamide (OEA).

In some embodiments, the Anandamide composition comprises, by weight, 35-70 parts of Anandamide, 6-12 parts of Linoleoylethanolamide, 3-15 parts of Oleoylethanolamide. Preferably, the Anandamide composition comprises, by weight, 36-68 parts of Anandamide, 8-11 parts of Linoleoylethanolamide, 4-13 parts of Oleoylethanolamide.

In some embodiments, the Anandamide composition further comprises, by weight, 0-20 parts of Palmitoylethanolamide (PEA), 0-25 parts of Stearoylethanolamide (SEA).

In some embodiments, the Anandamide composition further comprises, by weight, 0.01-15 parts of Palmitoylethanolamide, 0-20 parts of Stearoylethanolamide. In some embodiments, the Anandamide composition may further comprise, by weight, 0.05-13 parts, preferably 0.10-12 parts of Palmitoylethanolamide, 0-18 parts, preferably 0-15 parts of Stearoylethanolamide.

In some embodiments, the Anandamide composition further comprises, by weight, 2-8 parts of Octadecatrienoylethanolamide, 3-8 parts of Eicosatrienoylethanolamide, 0.8-3 parts of Eicosadienoylethanolamide, 0.01-3 parts of Tetradecanoylethanolamide. In some embodiments, the Anandamide composition further comprises, by weight, 2-8 parts of Octadecatrienoylethanolamide, 0.2-7 parts of Eicosatrienoylethanolamide, 0.8-3 parts of Eicosadienoylethanolamide, 0.01-3 parts of Tetradecanoylethanolamide. In some embodiments, the Anandamide composition may further comprise, by weight, 3-7 parts of Octadecatrienoylethanolamide, 4-7 parts of Eicosatrienoylethanolamide, 1-2.5 parts of Eicosadienoylethanolamide, 0.1-2.5 parts of Tetradecanoylethanolamide.

In some embodiments, the Anandamide composition may further comprise, by weight, 0-0.5 parts, preferably 0.01-0.4 parts of Eicosapentaenoylethanolamide, 0.1-1 parts, preferably 0.2-0.8 parts of Nonadecatetraenoylethanolamide, 0.3-1.5 parts, preferably 0.4-1 parts of Hexadecenoylethanolamide.

In some embodiments, the Anandamide composition may further comprise other suitable parts by weight of saturated or unsaturated ethanolamine.

In some embodiments, the Anandamide composition further comprises a dietetically or pharmaceutically acceptable carrier.

In some embodiments, the Anandamide composition is formulated as nutritional supplement, food, beverage, animal feed or medicine.

In some embodiments, the Anandamide composition is prepared into solid preparation or liquid preparation.

In some embodiments, the Anandamide composition is used for adjusting mood, relieving depression, relieving anxiety, improving cognitive ability and improving appetite.

In some embodiments, the Anandamide composition is in the form of suppository, tablet, pill, granule, powder, film, capsule, beverage, aerosol, elixir, tincture, tonic, liquid suspension or syrup.

In another aspect, the invention provides a method of using the Anandamide composition as described above, which includes administering the Anandamide composition to a subject in need thereof in an amount of 1 to 2000 mg per day. In some embodiments, the Anandamide composition is administered to a subject in need thereof in an amount of 0.01 to 6000 mg, 1 to 3000 mg, 5 to 1500 mg, 10 to 1200 mg, 15 to 1000 mg, or 30 to 800 mg per day.

The synthesis of AEA is not a one-step reaction, but needs multiple steps. Its synthesis conditions are harsh, which easily leads to the destruction of double bond structure and the generation of impurities with similar structures, which are mainly fatty acid ethanolamine, such as PEA, OEA, LEA and SEA. Because they are only slightly different in carbon chain length, double bond number and structure, and are very similar to AEA in physical and chemical properties, separation is extremely difficult. To separate them, it is necessary to use very precise purification techniques, such as preparative high performance liquid chromatography, and to repeatedly cool down, crystallize, filter and remove impurities. These methods can only handle a small number of samples at a time, which are expensive in equipment, time-consuming and solvent-consuming, resulting in an exponential increase in purification costs. For every 1 kg of AEA (AEA content >60%) produced in the actual production process, about 0.5 kg of solid by-products will be separated. Therefore, high-purity AEA can only exist as an expensive scientific research reagent, which greatly limits the market application of AEA. The composition of the invention develops a scheme of a composite formula, which saves the process of “cooling, crystallizing and filtering impurities” and greatly simplifies the preparation and purification process of the product.

### BRIEF DESCRIPTION OF THE FIGURES

FIG. 1 shows the immobility time of four groups of mice in the tail suspension test (TST).

FIG. 2 shows the immobility time of four groups of mice in the forced swimming test (FST).

### DETAILED DESCRIPTION OF THE INVENTION

Reference will now be made in detail to the preferred embodiments of the invention, examples of which are further illustrated. While the invention will be described in conjunction with the preferred embodiments, it will be understood that they are not intended to limit the invention to these embodiments. To the contrary, the invention is intended to cover alternatives, modifications and equivalents, which may be included within the spirit and scope of the invention as defined by the claims. Furthermore, in the detailed description of the present invention, numerous specific details are set forth in order to provide a thorough understanding of the present invention. However, it will be obvious to one of ordinary skill in the art that the present invention may be practiced without these specific details. In other instances, well known methods, procedures, components, and other features have not been described in detail as not to unnecessarily obscure aspects of the present invention.

As used herein, the term “or” is meant to include both “and” and “or.” In other words, the term “or” may also be replaced with “and/or.”

As used herein, the singular forms “a,” “an” and “the” are intended to include the plural forms as well, unless the context clearly indicates otherwise.

As used herein, the term “comprise” or “include” or their conjugations, refer to a situation where said terms are used in their non-limiting sense to mean that items following the word are included, but items not specifically mentioned are not excluded. It also encompasses the more limiting verb ‘to consist essentially of’ and ‘to consist of’.

As use herein, the terms “about” and “approximately” provide numerical flexibility by providing endpoints where a given value can be “slightly above” or “less than”. The flexibility of this term can be determined by specific variables and based on experience and related descriptions herein within the knowledge of those skilled in the art.

As used herein, the terms “subject” or “individual” are used interchangeably to refer to any subject to whom the disclosed compositions and methods can be applied or administered. The subject may have a disease or disorder, but the subject does not need to be sick to benefit from the disclosed methods and compositions. Subjects may need to improve their overall health, but they may also have overall healthy condition and wish to maintain or further improve their overall health. Therefore, any subject can take the disclosed composition or become a recipient of the disclosed method. As used herein, the term “subject” refers to animals (for example, birds, reptiles and mammals). In some embodiments, the subject can be mammals including non-primates (e.g., camels, donkeys, zebras, cows, horses, cats, dogs, rats and mice) and primates (e.g., monkeys, chimpanzees and humans). In certain embodiments, the subject may be a non-human mammal. In other embodiments, the subject may be a human.

As used herein, the term “administration” refers to the process of delivering a disclosed composition or active ingredient to a subject. The compositions of the invention can be administered in a variety of ways, including orally, intragastrically, and parenterally (e.g., intravenous and intraarterial as well as other suitable parenteral routes), and the like, so as to exert the desired effects.

As used herein, the term “dietetically or pharmaceutically acceptable” means pharmaceutically, physiologically, alimentarily, and/or nutritionally acceptable, and refers to those compositions or combinations of agents, materials, or compositions, and/or their dosage forms, which are within the scope of sound medical judgment, suitable for use in contact with the tissues of human beings and animals, compatible with other components of the composition, without excessive toxicity, irritation, allergic response, or other problem or complication, commensurate with a reasonable benefit/risk ratio.

Anandamide compositions will be formulated as a nutritional or dietary supplement, in a (medical) food item, in animal feed, or as a pharmaceutical composition in liquid or solid form comprising the composition of the invention, and may optionally also include a dietetically or pharmaceutically acceptable carrier. For example, where the composition is in solid form, the compositions may be formulated as a snack bar, yogurt, lozenge, tablet, or capsule, or may be coated onto cereal products, included in baked goods. On the other hand, where the supplement is in liquid form, the compositions may be formulated as a tincture, soft gel capsule, liquid capsule, syrup, carbonated drink, a brewed beverage (e.g., as coffee or tea), a juice, an energy drink, a sports drink, or flavored water. While nutritional and pharmaceutical compositions for human use are especially contemplated, it should be appreciated that the Anandamide composition and formulation thereof may also be employed for veterinary use (e.g., use in animal feed for domestic companion animals (‘pets’) or in animal feed for farm animals. In addition, the amount of the Anandamide composition to be added may vary according to the types and forms of nutritional supplements or dietary supplements, (medical) foods, animal feeds or pharmaceutical compositions.

The administration of the composition of the invention and preparation thereof can produce one or more desired effects, including but not limited to relieving pain, promoting sleep, stimulating appetite, improving concentration and memory, adjusting mood and relieving depression.

The following examples are illustrative of select embodiments of the present invention and are not meant to limit the scope of the invention.

### EXAMPLES

In order to make the purposes, technical schemes and advantages of the examples of the present invention clearer, the technical schemes in the examples will be described clearly and completely below. Where specific conditions are not specified in the examples, it shall be carried out according to the conventional conditions or the conditions suggested by the manufacturer. If the manufacturer is not indicated, the reagents or instruments used are all conventional products that can be purchased in the market.

### Preparation of Anandamide Composition

### Example 1

The composition of Example 1 comprises a mixture of about 30 parts of Anandamide (AEA), about 10 parts of Linoleoylethanolamide (LEA), about 18 parts of Oleoylethanolamide (OEA).

### Example 2

The composition of Example 2 comprises a mixture of about 35 parts of Anandamide (AEA), about 6 parts of Linoleoylethanolamide (LEA), about 15 parts of Oleoylethanolamide (OEA).

### Example 3

The composition of Example 3 comprises a mixture of about 45 parts of Anandamide (AEA), about 5 parts of Linoleoylethanolamide (LEA), about 13 parts of Oleoylethanolamide (OEA).

### Example 4

The composition of Example 4 comprises a mixture of about 62 parts of Anandamide (AEA), about 11 parts of Linoleoylethanolamide (LEA), about 4 parts of Oleoylethanolamide (OEA).

### Example 5

The composition of Example 5 comprises a mixture of about 65 parts of Anandamide (AEA), about 15 parts of Linoleoylethanolamide (LEA), about 5 parts of Oleoylethanolamide (OEA).

### Example 6

The composition of Example 6 comprises a mixture of about 70 parts of Anandamide (AEA), about 12 parts of Linoleoylethanolamide (LEA), about 3 parts of Oleoylethanolamide (OEA).

### Example 7

The composition of Example 7 comprises a mixture of about 75 parts of Anandamide (AEA), about 10 parts of Linoleoylethanolamide (LEA), about 2 parts of Oleoylethanolamide (OEA).

### Example 8

The composition of Example 8 comprises a mixture of about 30 parts of Anandamide (AEA), about 10 parts of Linoleoylethanolamide (LEA), about 18 parts of Oleoylethanolamide (OEA), about 15 parts of Palmitoylethanolamide (PEA), about 20 parts of Stearoylethanolamide (SEA).

### Example 9

The composition of Example 9 comprises a mixture of about 35 parts of Anandamide (AEA), about 6 parts of Linoleoylethanolamide (LEA), about 15 parts of Oleoylethanolamide (OEA), about 20 parts of Palmitoylethanolamide (PEA), about 25 parts of Stearoylethanolamide (SEA).

### Example 10

The composition of Example 10 comprises a mixture of about 42 parts of Anandamide (AEA), about 9 parts of Linoleoylethanolamide (LEA), about 13 parts of Oleoylethanolamide (OEA), about 12 parts of Palmitoylethanolamide (PEA), about 15 parts of Stearoylethanolamide (SEA).

### Example 11

The composition of Example 11 comprises a mixture of about 45 parts of Anandamide (AEA), about 5 parts of Linoleoylethanolamide (LEA), about 13 parts of Oleoylethanolamide (OEA), about 10 parts of Palmitoylethanolamide (PEA), about 10 parts of Stearoylethanolamide (SEA).

### Example 12

The composition of Example 12 comprises a mixture of about 62 parts of Anandamide (AEA), about 11 parts of Linoleoylethanolamide (LEA), about 4 parts of Oleoylethanolamide (OEA), about 0.1 parts of Palmitoylethanolamide (PEA).

### Example 13

The composition of Example 13 comprises a mixture of about 65 parts of Anandamide (AEA), about 15 parts of Linoleoylethanolamide (LEA), about 5 parts of Oleoylethanolamide (OEA), about 3 parts of Palmitoylethanolamide (PEA).

### Example 14

The composition of Example 14 comprises a mixture of about 70 parts of Anandamide (AEA), about 12 parts of Linoleoylethanolamide (LEA), about 3 parts of Oleoylethanolamide (OEA), about 1 part of Palmitoylethanolamide (PEA).

### Example 15

The composition of Example 15 comprises a mixture of about 75 parts of Anandamide (AEA), about 10 parts of Linoleoylethanolamide (LEA), about 2 parts of Oleoylethanolamide (OEA), about 0.01 parts of Palmitoylethanolamide (PEA).

### Example 16

The composition of Example 16 comprises a mixture of about 30 parts of Anandamide (AEA), about 10 parts of Linoleoylethanolamide (LEA), about 18 parts of Oleoylethanolamide (OEA), about 15 parts of Palmitoylethanolamide (PEA), about 20 parts of Stearoylethanolamide (SEA), about 4 parts of Octadecatrienoylethanolamide, about 3 parts of Eicosatrienoylethanolamide, about 3 parts of Eicosadienoylethanolamide, about 1 part of Tetradecanoylethanolamide.

### Example 17

The composition of Example 17 comprises a mixture of about 35 parts of Anandamide (AEA), about 6 parts of Linoleoylethanolamide (LEA), about 15 parts of Oleoylethanolamide (OEA), about 20 parts of Palmitoylethanolamide (PEA), about 25 parts of Stearoylethanolamide (SEA), about 2 parts of Octadecatrienoylethanolamide, about 5 parts of Eicosatrienoylethanolamide, about 1 part of Eicosadienoylethanolamide, about 0.01 parts of Tetradecanoylethanolamide.

### Example 18

The composition of Example 18 comprises a mixture of about 40 parts of Anandamide (AEA), about 8 parts of Linoleoylethanolamide (LEA), about 12 parts of Oleoylethanolamide (OEA), about 12 parts of Palmitoylethanolamide (PEA), about 15 parts of Stearoylethanolamide (SEA), about 3 parts of Octadecatrienoylethanolamide, about 4 parts of Eicosatrienoylethanolamide, about 2 parts of Eicosadienoylethanolamide, about 0.15 parts of Tetradecanoylethanolamide.

### Example 19

The composition of Example 19 comprises a mixture of about 60 parts of Anandamide (AEA), about 11 parts of Linoleoylethanolamide (LEA), about 4 parts of Oleoylethanolamide (OEA), about 0.1 parts of Palmitoylethanolamide (PEA), about 7 parts of Octadecatrienoylethanolamide, about 6 parts of Eicosatrienoylethanolamide, about 1 part of Eicosadienoylethanolamide, about 2 parts of Tetradecanoylethanolamide.

### Example 20

The composition of Example 20 comprises a mixture of about 70 parts of Anandamide (AEA), about 12 parts of Linoleoylethanolamide (LEA), about 3 parts of Oleoylethanolamide (OEA), about 1 part of Palmitoylethanolamide (PEA), about 8 parts of Octadecatrienoylethanolamide, about 8 parts of Eicosatrienoylethanolamide, about 0.8 parts f Eicosadienoylethanolamide, about 3 parts of Tetradecanoylethanolamide.

### Example 21

The composition of Example 21 comprises a mixture of about 75 parts of Anandamide (AEA), about 10 parts of Linoleoylethanolamide (LEA), about 2 parts of Oleoylethanolamide (OEA), about 0.01 parts of Palmitoylethanolamide (PEA), about 6 parts of Octadecatrienoylethanolamide, about 5 parts of Eicosatrienoylethanolamide, about 2 parts of Eicosadienoylethanolamide, about 1 part of Tetradecanoylethanolamide.

### Example 22

Example 22 comprises any of the compositions in Examples 1-21 and a dietetically or pharmaceutically acceptable carrier.

### Example 23 Establishment of Animal Model of Depression

Healthy male C57 mice, aged 7 weeks and weighing 18-22 g, which had been reared adaptively for more than one week, were selected. Mice were randomly divided into control group (n=6, group 1) and reserpine group (n=24). Mice in control group and reserpine group were injected with 0.1 mL/10 g saline or 0.5 mg/kg reserpine intraperitoneally for 8 days to establish an animal model of depression.

On the 9th day, the mice in reserpine group were further randomly divided into 3 groups (groups 2-4): Group 2 was injected intraperitoneally 0.5 mg/kg reserpine and soybean oil was given for 11 days. Group 3 was given 85 mg/kg of the composition of Example 12 by gavage and 0.5 mg/kg of reserpine by intraperitoneal injection for 11 days. Group 4 was given 85 mg/kg of the composition of Example 10 by gavage and 0.5 mg/kg of reserpine by intraperitoneal injection for 11 days. The first group continued to be injected with normal saline intraperitoneally. In the experiment, the composition was diluted with soybean oil. The mice were fasted for 2 hours before administration, and injected with normal saline or reserpine intraperitoneally 1 hour after administration.

### Example 24 Evaluation of Antidepressant Effect

### Tail Suspension Test (TST)

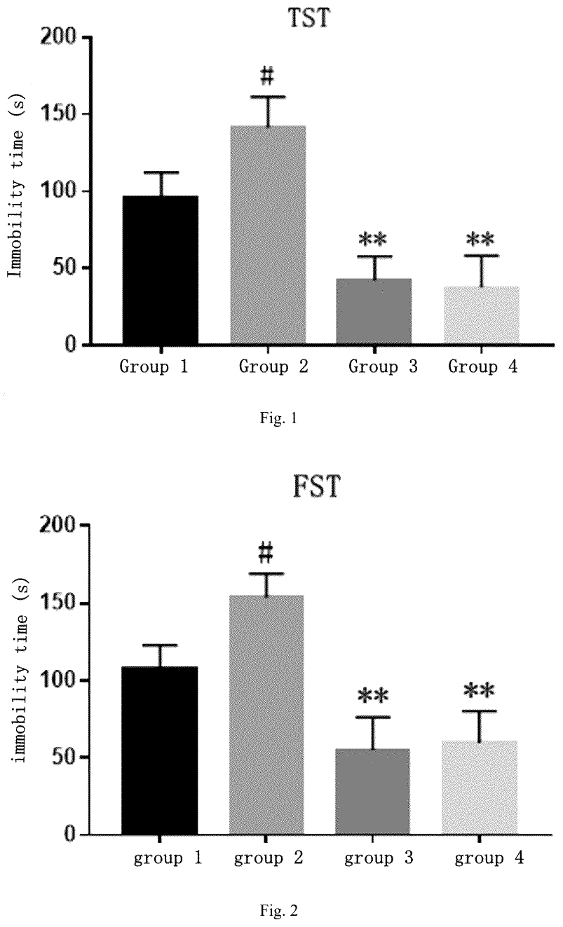

Mouse tail was each sticked with a medical tape (about 2 cm from the tail tip). Mouse was then hanged upside down on a tail suspension experimental device, with the head 15 cm away from the experimental platform, and the movement of the mouse was recorded with a video camera for 5 minutes. The immobility time of the mouse was counted within the next 4 min. FIG. 1 shows the immobility time of four groups of mice in the tail suspension test (TST).

### Forced Swimming Test (FST)

Mice were individually placed in a transparent cylindrical glass container (25 cm in height, 11 cm in diameter) filled with warm water (12 cm in water depth, 25±1° C. water temperature), and forced to swim. One mouse at a time in each container, and clean water should be replaced each time. The swimming of mouse was recorded with a video camera for 5 min, and the immobility time of mouse in the last 4 minutes was counted and recorded. FIG. 2 shows the immobility time of four groups of mice in the forced swimming test (FST).

In the tail suspension test (TST) and forced swimming test (FST), the immobility time of mice is an important index to judge whether the depression is relieved, and the immobility time is directly proportional to the depression degree of mice. As shown in FIGS. 1-2, there is a significant difference between group 2 (reserpine) and group 1 (control), indicating that the 8-day depression model was successfully established (# means there is a significant difference compared with the control, # means p<0.05; * means there is a significant difference compared with reserpine, ** means p<0.01). As shown in FIG. 1, the immobility time of mice in group 3 (reserpine+Example 12) and group 4 (reserpine+Example 10) was significantly shortened in the tail suspension test, and the composition of Example 12 and Example 10 reduced the immobility time of TST by36% and 39% respectively. As shown in FIG. 2, the immobility time of mice in group 3 and group 4 was significantly shortened in the forced swimming test, and the composition of Example 12 and Example 10 reduced the immobility time of FST by 29.8% and 27% respectively.

The Anandamide compositions of Examples 1-22 of the present invention have similar effects to those of Examples 12 and 10, and can effectively shorten the TST and FST immobility time of mice. Therefore, the composition of the invention can improve the depressive behavior of mice, has an antidepressant effect, and is a potential composition for adjusting mood, relieving depression, relieving anxiety, improving cognitive ability and improving appetite.

The above are only the preferred embodiments of the present invention, and are not intended to limit the present invention. Those skilled in the art can make many changes, modifications, substitutions and variations on these embodiments without departing from the principles and purposes of the invention, and the scope of the invention is defined by the claims and their equivalents.

What is claimed is:

1. An Anandamide composition comprising, by weight, 30-75 parts of Anandamide, 5-15 parts of Linoleoylethanolamide, 2-18 parts of Oleoylethanolamide.

2. The Anandamide composition of claim 1, wherein the Anandamide composition comprises, by weight, 35-70 parts of Anandamide, 6-12 parts of Linoleoylethanolamide, 3-15 parts of Oleoylethanolamide.

3. The Anandamide composition of claim 1, wherein the Anandamide composition comprises, by weight, 36-68 parts of Anandamide, 8-11 parts of Linoleoylethanolamide, 4-13 parts of Oleoylethanolamide.

4. The Anandamide composition of claim 1, wherein the Anandamide composition further comprises, by weight, 0-20 parts of Palmitoylethanolamide, 0-25 parts of Stearoylethanolamide.

5. The Anandamide composition of claim 1, wherein the Anandamide composition further comprises, by weight, 0.01-15 parts of Palmitoylethanolamide, 0-20 parts of Stearoylethanolamide.

6. The Anandamide composition of claim 1, wherein the Anandamide composition further comprises, by weight, 0.05-13 parts of Palmitoylethanolamide, 0-18 parts of Stearoylethanolamide.

7. The Anandamide composition of claim 1, wherein the Anandamide composition further comprises, by weight, 0.10-12 parts of Palmitoylethanolamide, 0-15 parts of Stearoylethanolamide.

8. The Anandamide composition of claim 1, wherein the Anandamide composition further comprises, by weight, 2-8 parts of Octadecatrienoylethanolamide, 3-8 parts of Eicosatrienoylethanolamide, 0.8-3 parts of Eicosadienoylethanolamide, 0.01-3 parts of Tetradecanoylethanolamide.

9. The Anandamide composition of claim 1, wherein the Anandamide composition further comprises, by weight, 2-8 parts of Octadecatrienoylethanolamide, 0.2-7 parts of Eicosatrienoylethanolamide, 0.8-3 parts of Eicosadienoylethanolamide, 0.01-3 parts of Tetradecanoylethanolamide.

10. The Anandamide composition of claim 1, wherein the Anandamide composition further comprises, by weight, 3-7 parts of Octadecatrienoylethanolamide, 4-7 parts of Eicosatrienoylethanolamide, 1-2.5 parts of Eicosadienoylethanolamide, 0.1-2.5 parts of Tetradecanoylethanolamide.

11. The Anandamide composition of claim 1, wherein the Anandamide composition further comprises, by weight, 0-0.5 parts of Eicosapentaenoylethanolamide, 0.1-1 parts of Nonadecatetraenoylethanolamide, 0.3-1.5 parts of Hexadecenoylethanolamide.

12. The Anandamide composition of claim 1, wherein the Anandamide composition further comprises, by weight, 0.01-0.4 parts of Eicosapentaenoylethanolamide, 0.2-0.8 parts of Nonadecatetraenoylethanolamide, 0.4-1 parts of Hexadecenoylethanolamide.

13. The Anandamide composition of claim 1, wherein the Anandamide composition further comprises a dietetically or pharmaceutically acceptable carrier.

14. The Anandamide composition of claim 1, wherein the Anandamide composition is formulated as nutritional supplement, food, beverage, animal feed or medicine.

15. The Anandamide composition of claim 1, wherein the Anandamide composition is prepared into solid preparation or liquid preparation.

16. The Anandamide composition of claim 1, wherein the Anandamide composition is used for adjusting mood, relieving depression, relieving anxiety, improving cognitive ability and improving appetite.

17. The Anandamide composition of claim 1, wherein the Anandamide composition is in the form of suppository, tablet, pill, granule, powder, film, capsule, beverage, aerosol, elixir, tincture, tonic, liquid suspension or syrup.
